(19) United States
(12) Patent Application Publication
(10) Pub. No.: US 2024/0285288 A1
(43) Pub. Date: Aug. 29, 2024

(54) Variable Angle Lapidus Cut Guide

(71) Applicant: Tyber Medical LLC, Bethlehem, PA (US)

(72) Inventors: Ryan C. Frasca, Easton, PA (US); Michael Gentile, Huntington, WV (US); Melissa D. Coale, Bethlehem, PA (US)

(21) Appl. No.: 18/692,477

(22) PCT Filed: Sep. 19, 2022
(86) PCT No.: PCT/US22/43927

§ 371 Date: Mar. 15, 2024

Related U.S. Application Data
(60) Provisional application No. 63/245,265, filed on Sep. 17, 2021.

(51) Int. Cl. A61B 17/15

(52) U.S. Cl. CPC A61B 17/151

(57) ABSTRACT

A variable angle osteotomy system is a de-rotation device capable of moving the first metatarsal toward the second metatarsal with a single hand, allowing the first metatarsal to be simultaneously rotated to proper anatomical position with the other hand. A de-rotation device that has a low profile and lays distal to first tarsometatarsal joint rests out of the surgeon's way while performing the osteotomy. A joint alignment feature allows the metatarsal cut slot to be placed at an exact distance from first tarsometatarsal joint. A feature allows alignment of the metatarsal cut guide to the long axis of first metatarsal. Two fixation elements to allow fixation of first metatarsal cut slot. A hinged attachment, between the metatarsal and cuneiform cut guides, allows rotation of the cuneiform cut slot relative to the fixated metatarsal cut slot. Two fixation elements allow fixation of the cuneiform cut slot to the cuneiform.

### CROSS-REFERENCE TO RELATED APPLICATION

The present application claims the benefit of U.S. Provisional Patent Application Ser. No. 63/245,265, filed on Sep. 17, 2021, which is incorporated herein by reference in its entirety.

### BACKGROUND OF THE INVENTION

### Field of the Invention

The invention relates to a Lapidus cut guide that can be adjusted to various angles depending on a patient's specific anatomy.

### Description of the Related Art

Lapidus cut guides are used to allow a clinician to make straight, accurate cuts at a metatarsal-cuneiform joint. Current guides, however, have fixed cutting angles and are unable to be adjusted based on the needs of a particular patient.

It would be beneficial to provide a variable angle cutting guide for performing an osteotomy on a patient's toe to provide for fixation of a metatarsal-cuneiform joint of a human foot.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a variable angle osteotomy system comprising an osteotomy guide having a metatarsal cut guide having a first cutting slot extending therethrough and an attachment arm and a cuneiform cut guide having a second cutting slot extending therethrough. The cuneiform cutting guide is pivotally attached to the attachment arm.

In another embodiment, the system further includes a de-rotation device comprising a first arm configured to attach to a second metatarsal and a second arm portion pivotally attached to the first arm. The second arm is configured to bias a first metatarsal toward the second metatarsal.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of an osteotomy guide according to an exemplary embodiment of the present invention;

FIG. 2 is another perspective view of the osteotomy guide of FIG. 1 illustrating a variable angle feature of the guide;

FIG. 3 is a front elevational view of the osteotomy guide of FIG. 1;

FIG. 4 is a right side elevational view of the osteotomy guide of FIG. 1;

FIG. 5 is a top plan view of the osteotomy guide of FIG. 1;

FIG. 6 is a left side elevational view of the osteotomy guide of FIG. 1;

FIG. 7 is a rear elevational view of the osteotomy guide of FIG. 1;

FIG. 8 is a perspective view of the cuneiform cut guide from the osteotomy guide of FIG. 1, taken along lines 9-9;

FIG. 9 is a cross section view of the osteotomy guide show in FIG. 8;

FIG. 10 is a perspective view of an osteotomy guide for the first Metatarsal;

FIG. 11 is another perspective view of an osteotomy guide for the first Metatarsal;

FIG. 12 is a perspective view of a first metatarsal de-rotation device;

FIG. 13 is another perspective view of the de-rotation device show in FIG. 12;

FIG. 14 is a perspective view of the first metatarsal attachment from the de-rotation device in FIG. 12;

FIG. 15 is another perspective view of the first metatarsal attachment from the de-rotation device in FIG. 12;

FIG. 16 is a perspective view of the second metatarsal attachment from the de-rotation device in FIG. 12;

FIG. 17 is a diagram of the required incisions for a procedure using the osteotomy guide and de-rotation device of FIGS. 1 and 12, respectively;

FIG. 18 is a perspective view of how the de-rotation device in FIG. 12 is intended to fit on a patient's foot;

FIG. 19 is a perspective view of an exemplary use of the de-rotation device in FIG. 12;

FIG. 20 shows a perspective view of one possible placement and use of the osteotomy guide in FIG. 1;

FIG. 21 shows a top plan view of the guide used in FIG. 20; and

FIG. 22 is a perspective view of pins inserted through the osteotomy device and the de-rotation device to stabilize toe members.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

The present invention provides a system for performing an osteotomy. The invention may be used to facilitate the cutting of the first metatarsal and a cuneiform allowing the fixation of the metatarsal-cuneiform joint of a human foot. The inventive system allows for an infinite variety of cut angles, depending on a particular patient's anatomy.

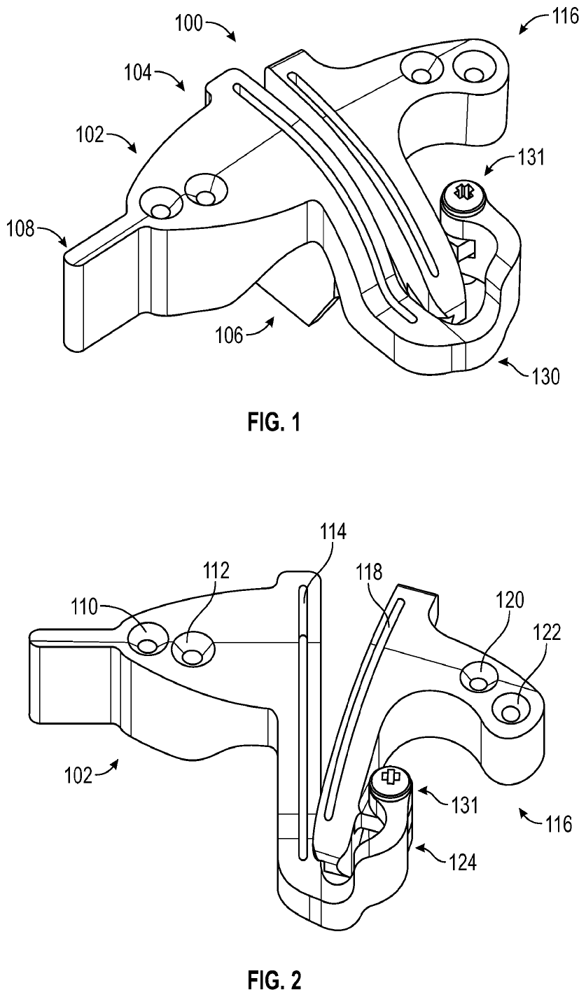

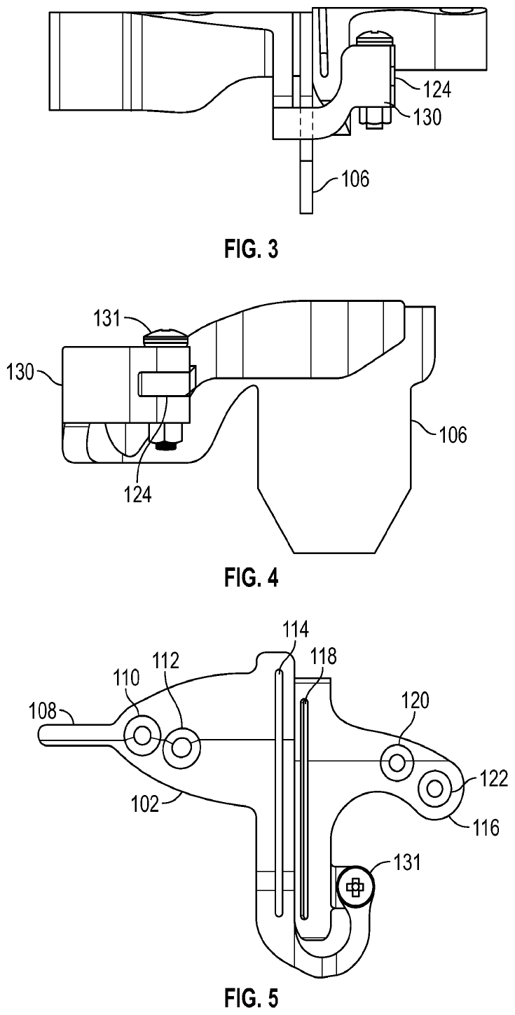

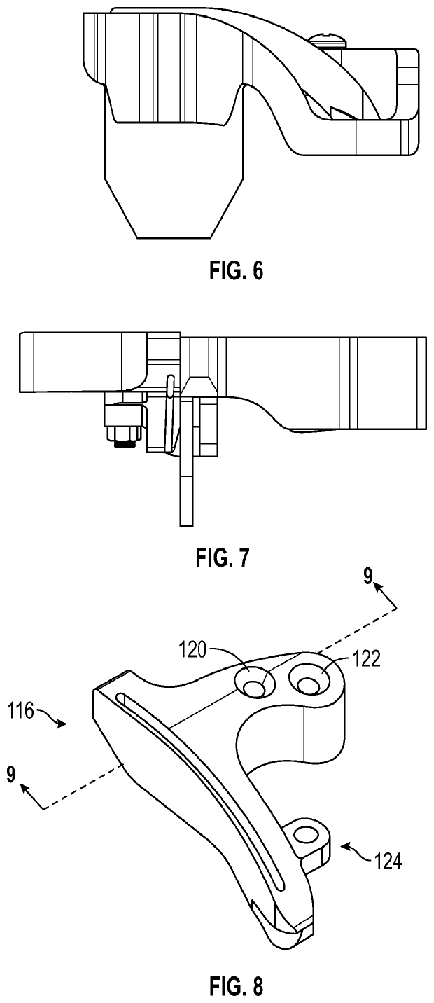

FIGS. 1-7 depict an osteotomy guide 100 that is composed of a metatarsal cut guide 102, which has a proximal end 104 which includes a protrusion 106. In an exemplary embodiment, protrusion 106 can be used as a joint spacer or to align the metatarsal cut guide 102. Metatarsal cut guide 102 also includes a distal protrusion 108 which, in an exemplary embodiment, can be used as a handle to align the metatarsal cut guide 102 parallel to the longitudinal axis of a first metatarsal Metatarsal cut guide 102 includes two fixation holes 110, and 112, which allow the guide 102 to be fixated to the first metatarsal using K-wires. Slot 114 on metatarsal cut guide 102 is intended to constrain the motion of a sagittal saw blade (not shown) as a cut is made in the first metatarsal. Osteotomy guide 100 also includes cuneiform cut guide 116. A slot 118 on the cuneiform cut guide 116 is intended to constrain the motion of the sagittal saw blade as a cut is made in the cuneiform. In an exemplary embodiment, cut guide 116 is able to be rotated relative to the metatarsal cut guide 102 to allow correction of first ray displacement in the sagittal plane. Cut guide 116 includes two fixation holes 120 and 122, which allow the guide 116 to be fixated to the cuneiform using K-wires.

FIG. 8 depicts a perspective view of the cuneiform cut guide 116. In an exemplary embodiment, attachment site 124 may be used to pivotally attach cut guides 102 and 116 to each other.

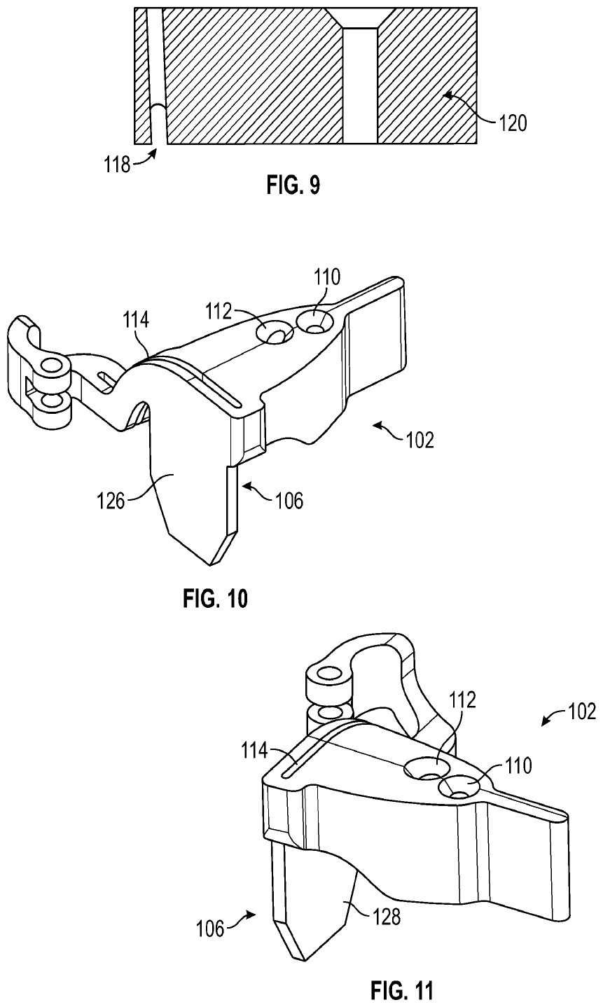

FIG. 9 depicts a cross section view of cuneiform cut guide 116. The 2-degree dorsal to plantar taper of slot 118 is shown here.

FIGS. 10 and 11 show perspective views of cut guide 102. In an exemplary embodiment, an optional protrusion 106 can be used as a joint spacer and alignment guide. Protrusion 106 is placed in the joint such that surface 126 is touching the distal end of the cuneiform and surface 128 is against the proximal end of the first metatarsal. This allows the cut guide 102 to be aligned such that slot 114 will guide the sagittal saw to take off a predetermined amount of bone from the first metatarsal. In an exemplary embodiment, attachment arm 130 can be used to attach the metatarsal cut guide 102 and the cuneiform cut guide 116 by connecting to the attachment site 124. Attachment arm 130 and attachment site 124 can be connected to each other via a securing member, such as a screw 131, screw 131 can be loosened to allow cut guide 102 to pivot relative to cuneiform cut guide 116 and tightened to secure cut guide 102 to cuneiform cut guide 116.

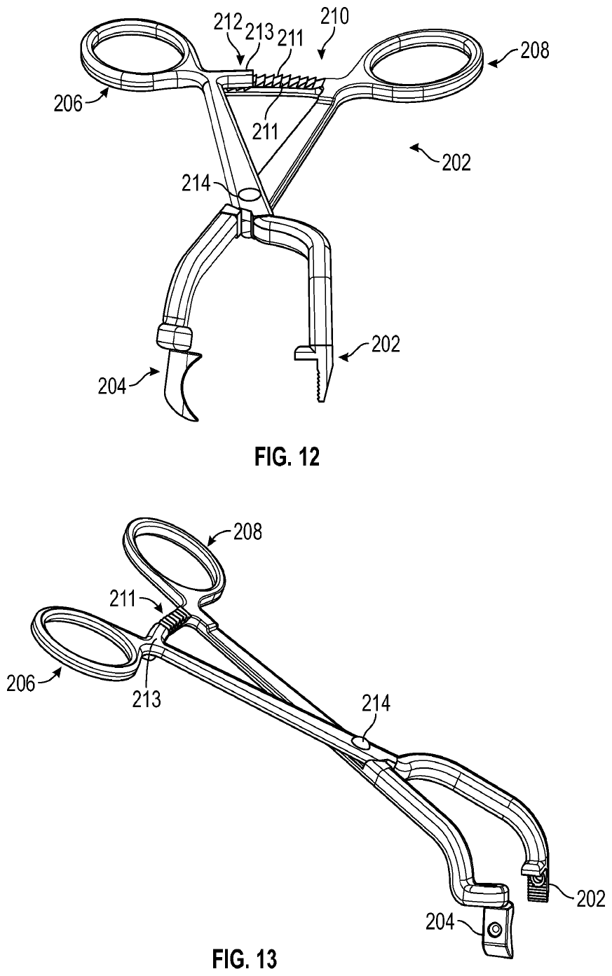

FIGS. 12-13 depict perspective views of a device 200 intended to be used for the de-rotation of the first metatarsal. An attachment portion 202 is intended to attach to the second metatarsal and provide a fixation point toward which the first metatarsal can be pushed or biased. The first metatarsal is intended to be pushed by metatarsal claw 204. In an exemplary embodiment, handle arms 206 and 208 can be used to bias metatarsal claw 204 toward attachment portion 202, compressing the first metatarsal toward the second metatarsal. Locking arm 210 includes known ratchet teeth 211 that can then be connected to locking arm 212 via an engaging member 213 to hold compression. Metatarsal claw 204 is pinned to a distal end of arm 208 by pin 214 to allow pivoting of metatarsal claw 204 relative to its arm 208. As metatarsal claw 204 is pivoted in in an arcuate movement as arms 206, 208 pivot about each other about pin 214, the pivoting of metatarsal claw 204 relative to its arm 208 ensures that the compression of the first metatarsal toward the second metatarsal will be linear.

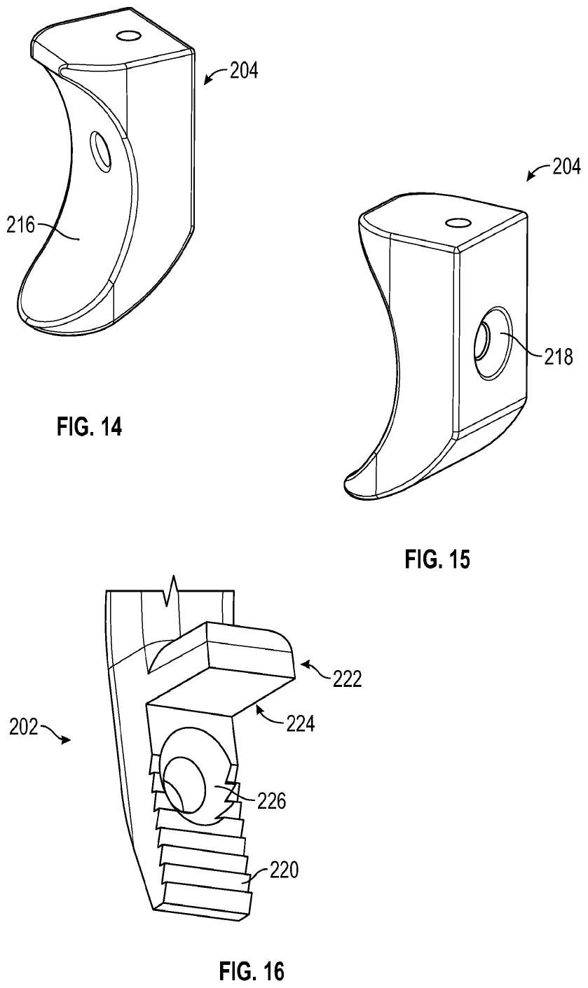

FIGS. 14-15 show the first metatarsal claw 204. In an exemplary embodiment, the claw 204 is intended to be placed on the medial side of the first metatarsal such that an arcuate surface 216 is engaging the first metatarsal and facing laterally. In an exemplary embodiment, a through-hole 218 can be used to guide a point-to-point K-wire.

FIG. 16 shows second metatarsal attachment portion 202 extending from a distal end of handle arm 206. In an exemplary embodiment, attachment portion 202 is intended to be placed between the second and 3^{rd} metatarsals with side surface 220 pressed against the lateral side of the second metatarsal. Side surface 220 includes a ribbed surface to enhance gripping the second metatarsal. A protrusion 222 extends outwardly from side surface 220 and can be used to tell when attachment 202 is far enough into the foot by placing lower surface 224 of protrusion 222 on top of the second metatarsal. In an exemplary embodiment, a through-hole 226 can be used to guide a point-to-point K-wire originally inserted through hole 218 in the metatarsal claw 204.

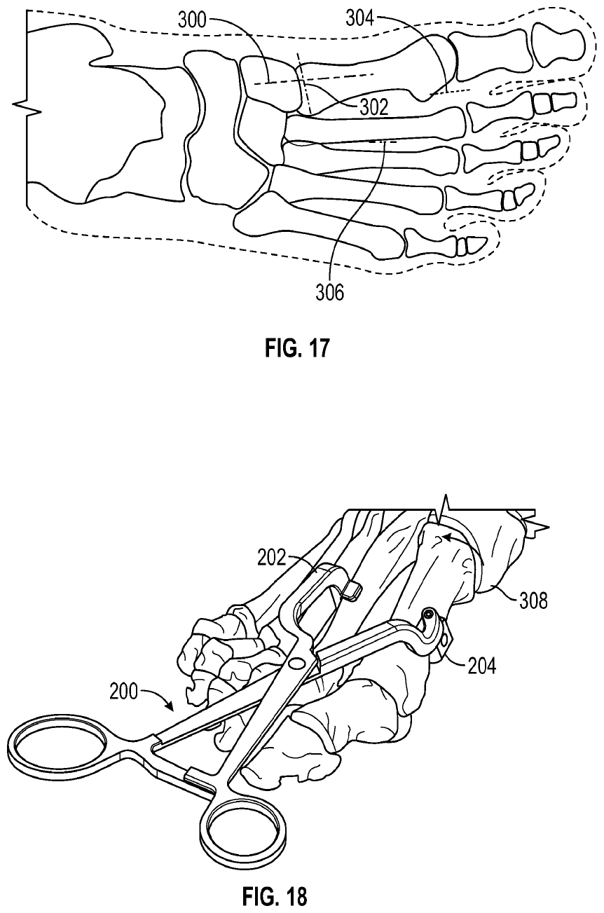

FIG. 17 shows the required incisions 300, 302, 304, 306 for an embodiment of this procedure. Incision 300 is a dorsal incision over the first tarsometatarsal (TMT) joint. Incision 302 is planning the TMT joint with a sagittal saw. The next incision 304 is meant to release the lateral capsule and suspensory ligaments. The final incision 306 is made for the second metatarsal attachment 202.

FIG. 18 depicts how de-rotation device 200 is intended to be placed on the foot. second metatarsal attachment 202 is placed through incision 306. Side surface 220 is placed against the lateral side of the second metatarsal and top surface 224 is placed on top of the second metatarsal. first metatarsal claw 204 is placed on the medial side of the first metatarsal. A k-wire 308 is inserted into the first metatarsal proximal to claw 204.

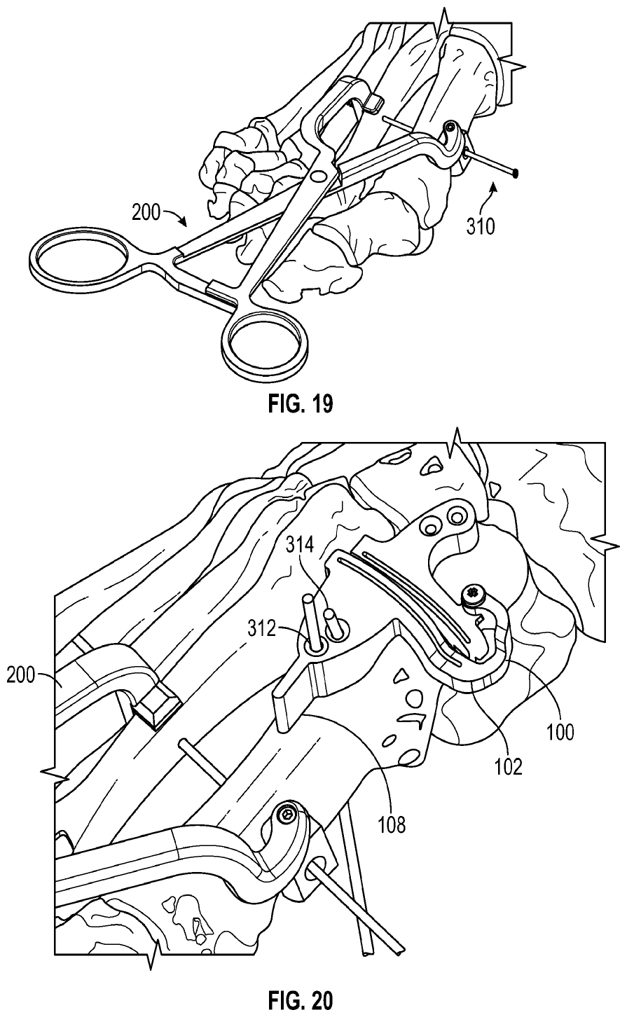

FIG. 19 shows a completed de-rotation of the first metatarsal. Device 200 is used to pull the first metatarsal toward the second metatarsal. At the same time K-wire 308 is rotated such that the sesamoids are returned to the appropriate anatomical position. A k-wire 310 is then inserted through through-hole 218 on the metatarsal claw 204. This K-wire 310 then is advanced through both the first metatarsal and second metatarsal, then into through-hole 226 on second metatarsal attachment 202. K-wire 310 ensures that corrected rotation is not lost during the rest of the procedure.

FIG. 20 shows the placement of the osteotomy guide 100 onto the first TMT joint. Protrusion 106 is inserted into the joint such that the metatarsal cut guide 102 is over the metatarsal. Protrusion 108 is then aligned with the long axis of the first metatarsal and k-wires 312 and 314 are inserted into holes 110 and 112 respectively to stabilize metatarsal cut guide 102 on the first metatarsal.

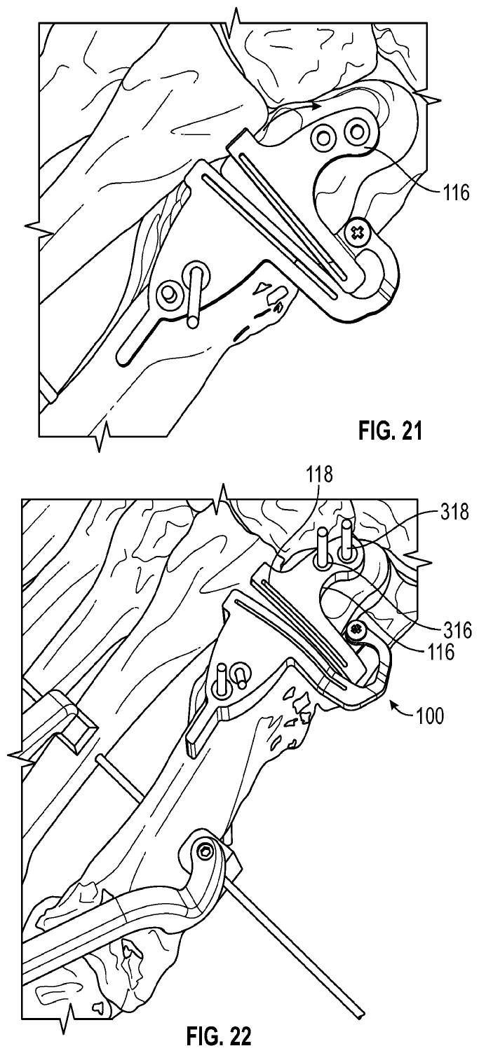

FIGS. 21-22 show the alignment of the cuneiform cut guide 116. The surgeon may pivot the cut guide 116 relative to the fixed cut guide 102 until cut slot 118 is perpendicular to their desired first metatarsal trajectory. K-wires 316 and 318 may then be inserted into holes 120 and 122 respectively to secure alignment. In one embodiment, cuts are then made using a sagittal saw in slots 114 and 118. All devices, equipment, and cut bone fragments are then removed and appropriate plates and screws can be implanted.

It will be further understood that various changes in the details, materials, and arrangements of the parts which have been described and illustrated in order to explain the nature of this invention may be made by those skilled in the art without departing from the scope of the invention as expressed in the following claims.

What is claimed is:

1. A variable angle osteotomy system comprising:
an osteotomy guide comprising:
a metatarsal cut guide having a first cutting slot extending therethrough and an attachment arm; and
a cuneiform cut guide having a second cutting slot extending therethrough, the cuneiform cutting guide being pivotally attached to the attachment arm.

2. The variable angle osteotomy system according to claim 1, wherein the metatarsal guide further includes a protrusion extending downwardly therefrom.

3. The variable angle osteotomy system according to claim 1, distal protrusion extending distally therefrom.

4. The variable angle osteotomy system according to claim 3, further comprising at least one metatarsal fixation hole extending therethrough between the first cutting slot and the distal protrusion.

5. The variable angle osteotomy system according to claim 1, further comprising at least one cuneiform fixation hole extending therethrough adjacent to the second cutting slot.

6. The variable angle osteotomy system according to claim 1, wherein the second cutting slot has a 2-degree dorsal to plantar taper formed therein.

7. The variable angle osteotomy system according to claim 1, further comprising a de-rotation device.

8. The variable angle osteotomy system according to claim 1, wherein the de-rotation device comprises:
a first arm configured to attach to a second metatarsal; and
a second arm portion pivotally attached to the first arm, the second arm configured to bias a first metatarsal toward the second metatarsal.

9. The variable angle osteotomy system according to claim 8, wherein the second arm comprises a metatarsal claw pivotally attached to a distal end thereof.

10. The variable angle osteotomy system according to claim 9, wherein the metatarsal claw comprises an arcuate face.

11. The variable angle osteotomy system according to claim 10, wherein the metatarsal claw has a through opening extending therethrough.

12. The variable angle osteotomy system according to claim 11, wherein the first arm has an attachment portion at a distal end thereof.

13. The variable angle osteotomy system according to claim 12, wherein the attachment portion has a ribbed surface.

14. The variable angle osteotomy system according to claim 12, wherein the attachment portion has a through opening extending therethrough.

15. The variable angle osteotomy system according to claim 14, wherein the through opening of the metatarsal claw and the through opening of the attachment portion are each sized to allow a single K-wire o be inserted therethrough.
